SEQUENCE LISTING (1 sequence)

SEQ ID NO: 1 - DNA, 20 bp, Artificial Sequence
  oligodeoxynucleotide (ODN) 1826
tccatgacgttcctgacgtt

(19) United States
(12) Patent Application Publication
(10) Pub. No.: US 2007/0071723 A1
(43) Pub. Date: Mar. 29, 2007

(54) In Vivo Enhancement of Immune System Recognition of Neoplasms Following Treatment with an Oncolytic Virus or Gene Therapy Vector

(75) Inventors: Matthew C. Coffey, Calgary, AB (CA); Bradley G. Thompson, Calgary, AB (CA)

Correspondence Address: FISH & RICHARDSON P.C., PO BOX 1022, MINNEAPOLIS, MN, 55440-1022, US

(73) Assignee: ONCOLYTICS BIOTECH INC., Calgary, AB (CA)

(21) Appl. No.: 11/468,676

(22) Filed: Aug. 30, 2006

Related U.S. Application Data
(60) Provisional application No. 60/713,287, filed on Aug. 31, 2005.

(51) Int. Cl. A61K 48/00; A61K 35/76

(52) U.S. Cl. 424/93.2; 424/93.6

(57) ABSTRACT

This invention provides novel methods of treating or alleviating neoplasms and enhancing the efficacy of oncolytic viruses by administering an oncolytic virus to a mammal suffering from a neoplasm and subsequently administering an immunostimulant. The invention also provides methods of increasing immunorecognition of neoplastic cells.

### CROSS-REFERENCE TO RELATED APPLICATION

This application claims priority to U.S. Provisional Application Ser. No. 60/713,287 filed Aug. 31, 2005. The disclosure of this application is incorporated herein by reference in its entirety.

### I. INTRODUCTION

A. Field of the Invention

This invention relates to methods of treating proliferative disorders in a mammal using oncolytic viruses and immunostimulants.

B. Background of the Invention

Cancer is diagnosed in more than 1 million people every year in the U.S. alone. In spite of numerous advances in medical research, cancer remains the second leading cause of death in the United States. In the industrialized nations, roughly one in five persons will die of cancer. In the search for novel strategies, oncolytic virus therapy has recently emerged as a viable approach to specifically kill tumor cells. Unlike conventional gene therapy, it uses replication competent viruses that are able to spread through tumor tissue by virtue of viral replication and concomitant cell lysis, providing an alternative treatment for cancer. Viruses have now been engineered to selectively replicate and kill cancer cells.

Oncolytic viruses may utilize multiple mechanisms of action to kill cancer cells—cell lysis, cell apoptosis, anti-angiogenesis and cell necrosis. The virus infects the tumor cell and then begins to replicate. The virus continues to replicate until finally “lyses” (bursts) the host cell's membrane as the tumor cell can no longer contain the virus. The tumor cell is destroyed and the newly created viruses are spread to neighboring cancer cells to continue the cycle. It is important to remember that all oncolytic viruses are intended to replicate only in cancer cells and to pass through normal tissue without causing harm. Hence, once all the tumor cells are eradicated, the oncolytic virus no longer has the ability to replicate and the immune system clears it from the body.

Over the past few years, new insights into the molecular mechanisms of viral cytotoxicity have provided the scientific rationale to design more effective oncolytic viruses. Recent advances in molecular biology have allowed the design of several genetically modified viruses, such as adenovirus and herpes simplex virus that specifically replicate in, and kill, tumor cells. On the other hand, viruses with intrinsic oncolytic capacity are also being evaluated for therapeutic purposes. Although the efficacy of oncolytic virus therapy in general has been demonstrated in preclinical studies, the therapeutic efficacy in clinical trails is still not optimal. Therefore, strategies are evaluated that could further enhance the oncolytic potential of conditionally replicating viruses.

### C. SUMMARY OF THE INVENTION

While it is recognized that administration of an oncolytic virus to a patient can elicit an antiviral immune response in the patient, the focus of research has been on circumventing this innate response. The present invention, on the other hand, takes advantage of this innate response to enhance killing of neoplasms. By administering immune stimulatory agents to patients following treatment with an oncolytic viral therapy, killing of the tumor cells can be increased. Not only are the tumor cells susceptible to the oncolytic virus, but also the infected tumor cells, which express viral antigen on their surface, can be recognized and attacked as ‘foreign’ by the stimulated immune system. Furthermore, tumor cells that have been lysed by the oncolytic virus are exposed to the immune system, thereby increasing the chance of immune system recognition of tumor antigens, particularly in the presence of immune stimulatory agents.

One aspect of the invention provides methods of treating a neoplasm in a mammal suffering from the neoplasm, the method comprising administering an oncolytic virus and an immunostimulant to the mammal. Preferably the immunostimulant is administered after the oncolytic virus, more preferably after the oncolytic virus has infected a neoplastic cell. Most preferably, the immunostimulant is administered after the infected neoplastic cell expresses at least one antigen of the oncolytic virus. Preferably, the immunostimulant is a synthetic oligodeoxynucleotide, such as cytosine-phosphate-guanosine (CpG). In a preferred embodiment, the oncolytic virus is a reovirus, more preferably a naturally-occurring reovirus.

In another aspect, the invention provides methods of enhancing the anti-neoplastic activity of an oncolytic virus in a mammal suffering from a neoplasm, the method comprising administering an immunostimulant in addition to administering the oncolytic virus to the mammal. Preferably, the immunostimulant is administered after the oncolytic virus is administered. More preferably, the immunostimulant is administered after the infected neoplastic cell expresses at least one antigen of the oncolytic virus. In an embodiment, the immunostimulant is a synthetic oligodeoxynucleotide (ODN), preferably unmethylated cytosine-phosphate-guanosine (CpG).

Yet another aspect of the invention provides methods of enhancing the anti-neoplastic activity of an oncolytic virus in a mammal suffering from said neoplasm, said method comprising (a) contacting a dendritic cell with the oncolytic virus, (b) inducing the dendritic cell to present an antigen of the oncolytic virus, and (c) eliciting an immune response to the antigen presented by the dendritic cell, thereby eliciting an immune response to the oncolytic virus in the mammal. In one preferred embodiment, step (a) occurs in vivo. In another preferred embodiment, step (a) occurs ex vivo and the dendritic cell is administered to the mammal after being contacted with the virus.

Another aspect of the invention provides a method of enhancing efficacy of an oncolytic virus therapy comprising administering an oncolytic virus to a mammal and administering an immunostimulant to the mammal. Preferably the immunostimulant is administered after the oncolytic virus, more preferably after the oncolytic virus has infected a neoplastic cell. Most preferably, the immunostimulant is administered after the infected neoplastic cell expresses at least one antigen of the oncolytic virus. Preferably, the immunostimulant is a synthetic oligodeoxynucleotide (ODN), such as cytosine-phosphate-guanosine (CpG). In a preferred embodiment, the oncolytic virus is a reovirus, more preferably a naturally-occurring reovirus.

An aspect of the invention provides methods of increasing immunorecognition of a neoplastic cell comprising (a) infecting the neoplastic cell with an oncolytic virus and (b) eliciting an immune response to an antigen of the oncolytic virus, whereby the immune response to the oncolytic virus responds to an oncolytic virus antigen expressed by the infected neoplastic cell. The immune response preferably is elicited by a process comprising (i) contacting a dendritic cell with the oncolytic virus, (ii) inducing the dendritic cell to present an antigen of the oncolytic virus and (iii) eliciting an immune response to the oncolytic virus. In one preferred embodiment, the contacting occurs in vivo. In another preferred embodiment, the contacting occurs ex vivo and the dendritic cell is administered to the mammal after contacting.

### II. DETAILED DESCRIPTION

### A. Definitions

“Administering” means any of the standard methods of administering a pharmaceutical composition known to those skilled in the art. Examples include, but are not limited to enteral, transdermal, intravenous, intramuscular or intraperitoneal administration. “Administration of a virus” to a subject refers to the act of administering the virus to a subject in a manner so that it contacts the target neoplastic cells. The route by which the virus is administered, as well as the formulation, carrier or vehicle, will depend on the location as well as the type of the target cells.

“Resistance” of cells to viral infection indicates that infection of the cells with the virus did not result in significant viral production or yield. Cells that are “susceptible” are those that demonstrate induction of cytopathic effects, viral protein synthesis, and/or virus production.

A “neoplastic cell,” “tumor cell,” or “cell with a proliferative disorder,” refers to a cell which proliferates at an abnormally high rate. A new growth comprising neoplastic cells is a neoplasm, also known as a “tumor.” A tumor is an abnormal tissue growth, generally forming a distinct mass, that grows by cellular proliferation more rapidly than normal tissue growth. A tumor may show partial or total lack of structural organization and functional coordination with normal tissue. As used herein, a tumor is intended to encompass hematopoietic tumors as well as solid tumors. A tumor may be benign (benign tumor) or malignant (malignant tumor or cancer). Malignant tumors can be broadly classified into three major types. Malignant tumors arising from epithelial structures are called carcinomas, malignant tumors that originate from connective tissues such as muscle, cartilage, fat or bone are called sarcomas and malignant tumors affecting hematopoietic structures (structures pertaining to the formation of blood cells) including components of the immune system, are called leukemias and lymphomas. Other tumors include, but are not limited to neurofibromatosis. The neoplastic cell is preferably located in a mammal, particularly a mammal selected from the group consisting of dogs, cats, rodents, sheep, goats, cattle, horses, pigs, human and non-human primates. Most preferably, the mammal is human.

An “oncolytic virus” is a virus that preferentially replicates in, and kills, neoplastic cells. An oncolytic virus may be a naturally-occurring virus or an engineered virus. Oncolytic viruses also encompass immunoprotected and reassortant viruses as described in detail for reovirus.

“Infection by an oncolytic virus” refers to the entry and replication of an oncolytic virus in a cell. Similarly, “infection of a tumor by an oncolytic virus” refers to the entry and replication of the oncolytic virus in the cells of the tumor.

An “effective amount” is an amount of an immunostimulant or reovirus which is sufficient to result in the intended effect. For an oncolytic virus used to treat or ameliorate a tumor, an effective amount is an amount of the oncolytic virus sufficient to alleviate or eliminate the symptoms of the tumor, or to slow down the progress of the tumor.

“Treating or alleviating a neoplasm” means alleviating or eliminating the symptoms of a neoplasm, or slowing down the progress of the neoplasm. The alleviation is preferably at least about 10%, more preferably at least about 20%, 30%, 40%, 50%, 60%, 70%, 80% or 90%.

The terms “nucleic acid” and “oligonucleotide” are used interchangeably to mean a molecule comprising multiple nucleotides. As used herein, the terms refer to oligoribonucleotides as well as oligodeoxyribonucleotides. The terms shall also include polynucleosides (i. e., a polynucleotide minus the phosphate) and any other organic base containing polymer. Nucleic acids include vectors, e.g., plasmids, as well as oligonucleotides. Nucleic acid molecules can be obtained from existing nucleic acid sources, but are preferably synthetic (e.g., produced by oligonucleotide synthesis).

An “immunostimulant” refers to essentially any substance that enhances or potentiates an immune response (antibody and/or cell-mediated) to an exogenous antigen.

An “immunostimulatory nucleic acid” as used herein is any nucleic acid containing an immunostimulatory motif or backbone that induces an immune response. The immune response may be characterized as, but is not limited to, a Th1-type immune response or a Th2-type immune response. Such immune responses are defined by cytokine and antibody production profiles which are elicited by the activated immune cells.

B. Methods of Treating Neoplasm

The invention provides methods of treating a neoplasm in a mammal suffering from said neoplasm, said method comprising administering an oncolytic virus and an immunostimulant to the mammal. The oncolytic virus is administered in a manner so that it can ultimately contact the target neoplastic cells. The route by which the oncolytic virus is administered, as well as the formulation, carrier or vehicle, will depend on the location as well as the type of the target cells. A wide variety of administration routes can be employed. For example, for a solid neoplasm that is accessible, the oncolytic virus can be administered by injection directly to the neoplasm. For a hematopoietic neoplasm, for example, the oncolytic virus can be administered intravenously or intravascularly. For neoplasms that are not easily accessible within the body, such as metastases, the oncolytic virus is administered in a manner such that it can be transported systemically through the body of the mammal and thereby reach the neoplasm (e.g., intravenously or intramuscularly). Alternatively, the oncolytic virus can be administered directly to a single solid neoplasm, where it then is carried systemically through the body to metastases. The oncolytic virus can also be administered subcutaneously, intraperitoneally, intrathecally (e.g., for brain tumor), topically (e.g., for melanoma), orally (e.g., for oral or esophageal neoplasm), rectally (e.g., for colorectal neoplasm), vaginally (e.g., for cervical or vaginal neoplasm), nasally or by inhalation spray (e.g., for lung neoplasm).

The oncolytic virus can be administered in a single dose, or multiple doses (i.e., more than one dose). The multiple doses can be administered concurrently at different sites or by different routes, or consecutively (e.g., over a period of days or weeks). The oncolytic virus is preferably administered prior to the immunosuppressant. In one embodiment of this invention, a course of virus/immunosuppressant therapy is administered one or more times.

The oncolytic virus is preferably formulated in a unit dosage form, each dosage containing from about 10² pfus to about 10¹³ pfus of the reovirus. The term “unit dosage forms” refers to physically discrete units suitable as unitary dosages for human subjects and other mammals, each unit containing a predetermined quantity of oncolytic virus calculated to produce the desired therapeutic effect, in association with a suitable pharmaceutical excipient.

The present invention can be applied to any animal subject, preferably a mammal. The mammal is preferably selected from the group consisting of canine, feline, rodent, domestic livestock (such as sheep, goats, cattle, horses, and pigs), human and non-human primates. Preferably, the mammal is human.

It is contemplated that the present invention may be combined with other tumor therapies such as chemotherapy, radiotherapy, surgery, hormone therapy and/or immunotherapy.

A person of ordinary skill in the art can practice the present invention using any oncolytic virus according to the disclosure herein and knowledge available in the art. The oncolytic virus may be a member in the family of myoviridae, siphoviridae, podpviridae, teciviridae, corticoviridae, plasmaviridae, lipothrixviridae, fuselloviridae, poxviridae, iridoviridae, phycodnaviridae, baculoviridae, herpesviridae, adenoviridae, papovaviridae, polydnaviridae, inoviridae, microviridae, geminiviridae, circoviridae, parvoviridae, hepadnaviridae, retroviridae, cyctoviridae, reoviridae, birnaviridae, paramyxoviridae, rhabdoviridae, filoviridae, orthomyxoviridae, bunyaviridae, arenaviridae, leviviridae, picomaviridae, sequiviridae, comoviridae, potyviridae, caliciviridae, astroviridae, nodaviridae, tetraviridae, tombusviridae, coronaviridae, glaviviridae, togaviridae, or barnaviridae.

Reoviruses are particularly preferred oncolytic viruses. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. The human reovirus consists of three serotypes: type 1 (strain Lang or T1L), type 2 (strain Jones, T2J) and type 3 (strain Dearing or strain Abney, T3D). The three serotypes are easily identifiable on the basis of neutralization and hemagglutinin-inhibition assays (see, for example, Fields, B. N. et al., 1996).

In another implementation of the invention, the oncolytic virus is an attenuated or modified adenovirus. Attenuated or modified adenovirus can replicate in cells with an activated Ras-pathway, but is unable to replicate in cells which do not have an activated Ras-pathway. Adenovirus is a double stranded DNA virus of about 3.6 kilobases. In humans, adenoviruses can replicate and cause disease in the eye and in the respiratory, gastrointestinal and urinary tracts. About one-third of the 47 known human serotypes are responsible for most cases of human adenovirus disease. The adenovirus encodes several gene products that counter antiviral host defense mechanisms. The virus-associated RNA (VAI RNA or VA RNA_{I}) of the adenovirus are small, structured RNAs that accumulate in high concentrations in the cytoplasm at late time after adenovirus infection. These VAI RNA bind to the double stranded RNA (dsRNA) binding motifs of PKR and block the dsRNA-dependent activation of PKR by autophosphorylation. Thus, PKR is not able to function and the virus can replicate within the cell. The overproduction of virions eventually leads to cell death. The term “attenuated adenovirus” or “modified adenovirus,” as used herein, means that the gene product or products which prevent the activation of PKR are lacking, inhibited or mutated such that PKR activation is not blocked. Preferably, the VAI RNA's are not transcribed. Such attenuated or modified adenovirus would not be able to replicate in normal cells that do not have an activated Ras-pathway, but it would be able to infect and replicate in cells having an activated Ras-pathway.

Newcastle disease virus (NDV) replicates preferentially in malignant cells, and the most commonly used strain is 73-T (Reichard et al., 1992; Zorn et al, 1994; Bar-Eli et al, 1996). PV701, an attenuated, non-recombinant, oncolytic strain of Newcastle disease virus, selectively lyses tumor cells versus normal cells based on tumor-specific defects in an interferon-mediated antiviral response.

Parapoxvirus orf virus is a poxvirus that induces acute cutaneous lesions in different mammalian species, including humans. The parapoxvirus orf virus encodes the gene OV20.0L that is involved in blocking PKR activity. The parapoxvirus orf virus is unable to replicate in cells that do not have an activated Ras-pathway. A more preferred oncolytic virus for use in the invention is an “attenuated parapoxvirus orf virus” or “modified parapoxvirus orf virus,” in which the gene product or products which prevent the activation of PKR are lacking, inhibited or mutated such that PKR activation is not blocked. Preferably, the gene OV20.0L is not transcribed. Such attenuated or modified parapoxvirus orf virus would not be able to replicate in normal cells that do not have an activated Ras-pathway, but it is able to infect and replicate in cells having an activated Ras-pathway.

A herpes simplex virus 1 (HSV-1) mutant which is defective in ribonucleotide reductase expression, hrR3, was shown to replicate in colon carcinoma cells but not normal liver cells (Yoon et al., 2000). Herpes simplex virus type 1 (HSV-1) vectors are particularly useful, because they can be genetically engineered to replicate and spread highly selectively in tumor cells and can also express multiple foreign transgenes. These vectors can manifest a cytopathic effect in a wide variety of tumor types without damaging normal tissues, provide amplified gene delivery within the tumor, and induce specific antitumor immunity. Multiple recombinant HSV-1 vectors have been tested in patients with brain tumors and other cancers, which showed the feasibility of administering replication-competent HSV-1 vectors safely in human organs including the brain.

Many other oncolytic viruses are known to those of skill in the art. For example, vesicular stomatitis virus (VSV) selectively kills neoplastic cells. Encephalitis virus was shown to have an oncolytic effect in a mouse sarcoma tumor, but attenuation may be required to reduce its infectivity in normal cells. Vaccinia virus, due to its exceptional ability to replicate in tumor cells, represents another replicating oncolytic virus useful in the present invention. In addition, specific viral functions can be augmented or eliminated to enhance anti-tumor efficacy and improve tumor cell targeting. For example, the deletion of viral genes for thymidine kinase and vaccinia growth factor result in vaccinia mutants with enhanced tumor targeting activity. In a preferred implementation, the oncolytic virus is a modified vaccinia virus, as described in U.S. patent publication No. 2002/0028195, in which E3L or K3L is mutated. The vaccine strain of measles virus (MV) readily lyses transformed cells, while replication and lysis are limited in normal human cells. Thus, MV is highly suitable for development as an oncolytic agent. Tumor regression also has been described in tumor patients infected with herpes zoster, hepatitis virus, influenza, varicella, and measles virus (for a review, see Nemunaitis, 1999). Any oncolytic virus may be used in the claimed invention.

The ability of various oncolytic viruses to replicate selectively in neoplastic cells is known to rely on different mechanisms. Reovirus, for example, requires the presence of an activated Ras signaling pathway in order to replicate and destroy cells. In some other oncolytic viruses, tumor selectivity is achieved by placing an essential viral gene under the control of a tumor-specific promoter. In certain viruses, the E1A region is responsible for binding to the cellular tumor suppressor Rb and inhibiting Rb function, thereby allowing the cellular proliferative machinery, and hence virus replication, to proceed in an uncontrolled fashion. Delta24 has a deletion in the Rb binding region and does not bind to Rb (Fueyo et al., 2000). Therefore, replication of the mutant virus is inhibited by Rb in a normal cell. However, if Rb is inactivated and the cell becomes neoplastic, Delta24 is no longer inhibited. Thus, the mutant virus replicates efficiently and lyses Rb-deficient neoplastic cells. Other mechanisms for selective replication in neoplastic cells are known in the art. The present invention places no limitation on the mechanism by which the oncolytic virus replicates selectively in neoplastic cells as compared to normal cells.

It is preferable that the virus is not a vehicle for delivering a gene for the purpose of gene therapy. For example, viruses have been engineered to deliver the adenoviral E1A gene, the p53 tumor suppressor gene, prodrug-encoding genes (Chmura et al., 1999; 2001) or genes under a radiation-inducible promoter. These viruses, in fact, usually do not replicate preferentially in neoplastic cells and therefore would not be considered oncolytic viruses.

The oncolytic virus may be naturally occurring or modified. The oncolytic virus is “naturally-occurring” when it can be isolated from a source in nature and has not been intentionally modified by humans in the laboratory. For example, the oncolytic virus can be from a “field source,” that is, from a human who has been infected with the oncolytic virus.

The oncolytic virus may be a recombinant oncolytic virus resulting from the recombination/reassortment of genomic segments from two or more genetically distinct oncolytic viruses. Recombination/reassortment of oncolytic virus genomic segments may occur in nature following infection of a host organism with at least two genetically distinct oncolytic virus. Recombinant virions can also be generated in cell culture, for example, by co-infection of permissive host cells with genetically distinct oncolytic viruses (Nibert et al. 1995). The invention further contemplates the use of recombinant oncolytic virus resulting from reassortment of genome segments from two or more genetically distinct oncolytic viruses wherein at least one parental virus is genetically engineered, comprises one or more chemically synthesized genomic segment, has been treated with chemical or physical mutagens, or is itself the result of a recombination event. The invention further contemplates the use of the recombinant oncolytic virus that has undergone recombination in the presence of chemical mutagens, including but not limited to dimethyl sulfate and ethidium bromide, or physical mutagens, including but not limited to ultraviolet light and other forms of radiation.

The invention further contemplates the use of recombinant oncolytic viruses that comprise deletions or duplications in one or more genome segments, that comprise additional genetic information as a result of recombination with a host cell genome, or that comprise synthetic genes.

The oncolytic virus may be modified but still capable of lytically infecting a neoplastic mammalian cell. The oncolytic virus may be chemically or biochemically pretreated (e.g., by treatment with a protease, such as chymotrypsin or trypsin) prior to administration to the proliferating cells. Pretreatment with a protease can remove the outer coat or capsid of the virus and may increase the infectivity of the virus. The oncolytic virus may be coated in a liposome or micelle. For example, the virion may be treated with chymotrypsin in the presence of micelle forming concentrations of alkyl sulfate detergents to generate a new infectious subvirion particle.

The oncolytic virus may be modified by incorporation of mutated coat proteins, such as for example, into the virion outer capsid. The proteins may be mutated by replacement, insertion or deletion. Replacement includes the insertion of different amino acids in place of the native amino acids. Insertions include the insertion of additional amino acid residues into the protein at one or more locations. Deletions include deletions of one or more amino acid residues in the protein. Such mutations may be generated by methods known in the art. For example, oligonucleotide site directed mutagenesis of the gene encoding for one of the coat proteins could result in the generation of the desired mutant coat protein. Expression of the mutated protein in oncolytic virus infected mammalian cells in vitro such as COS 1 cells can result in the incorporation of the mutated protein into the oncolytic virus virion particle (Turner and Duncan, 1992; Duncan et al., 1991; Mah et al., 1990).

One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A, QS21, aminoalkyl glucosaminide 4-phosphates, and quil A. Cytokines, such as GM-CSF, interleukin-2, -7, -12, and other like growth factors, may also be used as adjuvants.

The immunostimulant is administered to the host in the manner conventional for the particular composition, generally as a single unit dose in buffered saline. Optionally booster doses, typically one to several weeks later, can additionally be delivered enterally or parenterally, e.g., subcutaneously, cutaneously, intramuscularly, intradermally, intravenously, intraarterially, intraperitoneally, intranasally, orally, intraheart, intrapancreas, intraarticular, etc. Localization of the initial or booster dose of immunostimulant can be achieved by administration at the targeted site, use of sustained release implants, delivery in the form of non-diffusible particles, and the like, as known in the art. The dose and protocol for delivery of the immunostimulant will vary with the specific agent that is selected. Typically one or more doses are administered.

In one embodiment of the invention, the immunostimulant is a polyclonal activating agent, which may include endotoxins, e.g., lipopolysaccharide (LPS); and superantigens (exotoxins) (see Herman et al. (1991) Annu Rev Immunol 9:745-72). Endotoxin primarily interacts with CD14 receptors on macrophages, while superantigens preferentially activate T cells. Both cell types are thus triggered to release pro-inflammatory cytokines. Superantigens (SAgs) are presented by major histocompatibility complex (MHC) class II molecules and interact with a large number of T cells expressing specific T cell receptor V beta domains.

Alternatively, one may use immunostimulatory nucleic acids. Immunostimulatory nucleic acids may possess immunostimulatory motifs such as CpG motif, and poly-G motifs. In some embodiments of the invention, any nucleic acid, regardless of whether it possesses an identifiable motif, can be used in the combination therapy to elicit an immune response. In one embodiment, the immunostimulatory nucleic acid contains the sequence CpG, preferably a consensus mitogenic CpG motif represented by the formula: 5′ X₁X₂CGX₃X₄ 3′, where C and G are unmethylated, X₁, X₂, X₃ and X₄ are nucleotides and a GCG trinucleotide sequence is not present at or near the 5′ and 3′ termini (see U.S. Pat. No. 6,008,200, Krieg et al., issued Dec. 28, 1999). CpG immunostimulatory nucleic acids are known to stimulate Th1-type immune responses. CpG sequences, while relatively rare in human DNA, are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG containing nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570, PCT/US97/19791, PCT/US98/03678, PCT/US98/10408, PCT/US98/04703, PCT/US99/07335, and PCT/US99/09863.

In another embodiment, the immunostimulatory nucleic acids are poly-G immunostimulatory nucleic acids. A variety of references, including Pisetsky and Reich, 1993 Mol Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 describe the immunostimulatory properties of poly-G nucleic acids.

The immunostimulatory nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded. The entire immunostimulatory nucleic acid, or portions thereof, can be unmethylated, but at least the C of the 5′ CpG 3′ must be unmethylated.

For facilitating uptake into cells, the immunostimulatory nucleic acids are preferably in the range of 2 to 100 bases in length. However, nucleic acids of any size greater than 6 nucleotides (even many kb long) are capable of inducing an immune response if sufficient immunostimulatory motifs are present. Preferably the immunostimulatory nucleic acid is between 8 and 100 nucleotides, and in some embodiments, between 8 and 50 or 8 and 30 nucleotides in size.

One particular advantage of the use of immunostimulatory nucleic acids in the methods of the invention is that immunostimulatory nucleic acids can exert immunomodulatory activity even at relatively low dosages. Although the dosage used will vary depending on the clinical goals to be achieved, a suitable dosage range is one which provides from about 1 Fg to about 10,000 Fg, usually at least about 1,000 Fg of immunostimulatory nucleic acids, in a single dosage. Alternatively, a target dosage of immunostimulatory nucleic acids results in about 1-10 femtomolar of immunostimulatory nucleic acid in a volume of host blood drawn within the first 24-48 hours after administration of the immunostimulatory nucleic acids. Based on current studies, immunostimulatory nucleic acids are believed to have little or no toxicity at these dosage levels.

Immunostimulatory nucleic acids suitable for the purposes of the invention can be in the form of phosphodiesters or, in order to be more stable, in the form of phosphorothioates or of phosphodiester/phosphorothioate hybrids. Although it is possible to use oligonucleotides originating from existing nucleic acid sources, such as genomic DNA or cDNA, preference is given to the use of synthetic oligonucleotides. Thus, it is possible to develop oligonucleotides on a solid support using the β-cyanoethyl phosphoramidite method (Beaucage, S. L. and Caruthers, M. H. Tetrahedron Letters 22, 1859-1862 (1981)) for the 3′→5′ assembly, and then precipitation in ethanol in the presence of 0.3 M sodium acetate not adjusted for pH (0.3M final) is carried out. Next, precipitation with 4 volumes of 80% ethanol is carried out, followed by, drying before taking up the precipitate in pure water. In the phosphorothioate-containing oligonucleotides, one of the oxygen atoms making up the phosphate group is replaced with a sulfur atom. Their synthesis can be carried out as previously described, except that the iodine/water/pyridine tetrahydrofuran solution which is used in the oxidation step required for the synthesis of the phosphodiester linkages is replaced with a TETD (tetraethylthiuram disulfide) solution, which provides the sulfate ions for the production of the phosphorothioate group. It is also possible to envisage other modifications of the phosphodiester linkages, of the bases or of the sugars, so as to modify the properties of the oligonucleotides used in particular to increase their stability.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. Immunostimulatory backbones include, but are not limited to, phosphate modified backbones, such as phosphorothioate backbones. The use of these immunostimulatory sequences is known in the art, for examples see Bauer et al. (1999) Immunology 97(4):699-705; Klinman et al. (1999) Vaccine 17(1):19-25; Hasan et al. (1999) J Immunol Methods 229(1-2):1-22; and others. One type of such a modification is a phosphate backbone modification. For example, immunostimulatory nucleic acids, including at least two phosphorothioate linkages at the 5′ end of the oligonucleotide and multiple phosphorothioate linkages at the 3′ end (preferably 5), can provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, and combinations thereof. Each of these combinations in immunostimulatory nucleic acids and their particular effects on immune cells is discussed in more detail in PCT Published Patent Applications PCT/US95/01570 and PCT/US97/19791.

Preferred immunostimulants for eliciting a predominantly Th1-type response include, for example, a combination of monophosphoryl lipid A, preferably 3-de-O-acylated monophosphoryl lipid A together with an aluminum salt. CpG-containing oligonucleotides (in which the CpG dinucleotide is unmethylated) also induce a predominantly Th1 response. Another preferred immunostimulant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin, for example combinations of at least two members selected from one group consisting of QS21, QS7, Quil A, βescin, and digitonin.

According to another embodiment of this invention, the immunostimulant is at least one antigen of an oncolytic virus delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response. APCs may generally be isolated from any of a variety of biological fluids and organs, including tumor and peritumoral tissues, and may be autologous, allogeneic, syngeneic or xenogeneic cells.

Cancer immunotherapy using dendritic cells loaded with tumor-associated antigens have been shown to produce tumor-specific immune responses and anti-tumor activity (Campton et al. 2000; Fong and Engelmann 2000). Promising results were obtained in clinical trials in vivo using tumor antigen pulsed dendritic cells (Tarte and Klein 1999). These studies clearly demonstrate the efficacy of using dendritic cells to generate immune responses against cancer antigens.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529,1999). In general, dendritic cells may be identified based on their typical shape (stellate in situ, with marked cytoplasmic processes (dendrites) visible in vitro), their ability to take up, process and present antigens with high efficiency, and their ability to activate nave T cell responses. Dendritic cells may be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used (see Zitvogel et al., Nature Med. 4:594-600,1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, fit3 ligand and/or other compound(s) that induce differentiation, maturation and proliferation of dendritic cells.

### III. EXAMPLE

### Example 1

Two groups of female SCID mice are injected with 1×10⁶ human breast carcinoma MDA-MB468 cells in two subcutaneous sites, overlying both hind flanks. Palpable tumors are evident approximately two to four weeks post injection. Undiluted reovirus serotype three (strain Dearing) is injected into the right side tumor mass in a volume of 20 μl at a concentration of 1.0×10⁷ PFU/ml. Animals in group one also are injected with 10 μg of ODN 1826 (TCCATGACGTTCCTGACGTT), a CpG-containing oligonucleotide, along with the reovirus. Two weeks later, these animals are injected again with the same amount of ODN 1826. Animals in group two receive saline injections in the same amount and same frequency as the CpG. The results show that in both groups, the size of the tumors on the left side of animals is greater than the size of the tumors on the right side of the animals, indicating that oncolytic virus therapy is effective in treating neoplasms. Further, the size of tumors in the left side of animals in group one is smaller than the size of tumors in the left side of animals in group two, indicating the additional anti-tumor effect of administering immunostimulant in conjunction with an oncolytic virus therapy.

We claim:

1. A method of treating or alleviating a neoplasm in a mammal suffering from said neoplasm, said method comprising:
(a) administering an oncolytic virus to the mammal; and
(b) administering an immunostimulant.

2. The method of claim 1, wherein the immunostimulant is administered after the oncolytic virus.

3. The method of claim 2, wherein the immunostimulant is administered after the oncolytic virus has infected a tumor cell.

4. The method of claim 3, wherein the immunostimulant is administered after the infected tumor cell expresses at least one antigen of the oncolytic virus or a tumor-specific antigen.

5. The method of claim 2, wherein the immunostimulant is administered 24 hours after the oncolytic virus.

6. The method of claim 1, wherein the oncolytic virus is a reovirus.

7. The method of claim 6, wherein the reovirus is a naturally-occurring reovirus.

8. The method of claim 1, wherein the immunostimulant is a synthetic oligodeoxynucleotide (ODN).

9. The method of claim 8, wherein the immunostimulant is unmethylated cytosine-phosphate-guanosine (CpG).

10. A method of enhancing the anti-neoplastic activity of an oncolytic virus in a mammal suffering from a neoplasm, said method comprising administering an immunostimulant and an oncolytic virus to the mammal.

11. The method of claim 10, wherein the immunostimulant is administered after the oncolytic virus has infected a tumor cell.

12. The method of claim 10, wherein the immunostimulant is administered after the infected cell expresses at least one antigen of the oncolytic virus or a tumor-specific antigen.

13. The method of claim 11, wherein the immunostimulant is administered 24 hours after the oncolytic virus.

14. The method of claim 10, wherein the oncolytic virus is a reovirus.

15. The method of claim 14, wherein the reovirus is a naturally-occurring reovirus.

16. The method of claim 10, wherein the immunostimulant is a synthetic oligodeoxynucleotide (ODN).

17. The method of claim 16, wherein the immunostimulant is unmethylated cytosine-phosphate-guanosine.

18. A method of enhancing the anti-neoplastic activity of an oncolytic virus in a mammal suffering from a neoplasm, said method comprising:
(a) contacting a dendritic cell with the oncolytic virus;
(b) inducing the dendritic cell to present an antigen of the oncolytic virus; and
(c) eliciting an immune response to the oncolytic virus in the mammal.

19. The method of claim 18, wherein the contacting occurs ex vivo and the dendritic cell is administered to the mammal after contacting.

20. A method of enhancing efficacy of an oncolytic virus therapy comprising:
(a) administering an oncolytic virus to a mammal; and
(b) administering an immunostimulant.

21. The method of claim 20, wherein the immunostimulant is administered after the oncolytic virus.

22. The method of claim 21, wherein the immunostimulant is administered after the oncolytic virus has infected a tumor cell.

23. The method of claim 21, wherein the immunostimulant is administered 24 hours after the oncolytic virus therapy.

24. The method of claim 20, wherein the oncolytic virus therapy is a reovirus.

25. The method of claim 24, wherein the reovirus is a naturally-occurring reovirus.

26. The method of claim 20, wherein the immunostimulant is a synthetic oligodeoxynucleotide (ODN).

27. The method of claim 26, wherein the immunostimulant is unmethylated cytosine-phosphate-guanosine.

28. A method of increasing immunorecognition of a neoplastic cell comprising:
(a) infecting the neoplastic cell with an oncolytic virus;
(b) eliciting an immune response to an antigen of the oncolytic virus by a process comprising:
(i) contacting a dendritic cell with the oncolytic virus;
(ii) inducing the dendritic cell to present an antigen of the oncolytic virus; and
(iii) eliciting an immune response to the oncolytic virus;
whereby the immune response to the oncolytic virus responds to an oncolytic virus antigen expressed by the infected neoplastic cell.
